SEQUENCE LISTING (26 sequences)

SEQ ID NO: 1 - DNA, 645 bp, Porcine circovirus
ttagagaacggacttgtaacgaatccaaacttctttcgtgccgtagaagtctgtcattccagttttttccgggacataaatgctccaaagcagtgctccccattgaacggtggggtcatatgtgttgagccatggggtgggtctggagaaaaagaagaggctttgtcctgggtgagcgctggtagttcccgccagaattggtttcggggtgaagtaacggctgtgtttttttttagaagtcataactttacgagtggaactttccgcataagggtcgtcttggagccaagtgtttgtggtccaggcgccgtctagatctatggctgtgtgcccgaacatagtttttgtttgctgagctggagaaattacagggctgagtgtaactttcatctttagtatcttataatattcaaagctaattgcagtttcccactcgtttaggcgggtaatgaagtggttggcgtgccagggcttattattctgaggagttccaacggaaatgacgttcatggtggagtatttctttgtgtagtatgtgccagctgtgggcctcctaatgaatagtcttcttctggcatagcgccttctgtggcgtcgtcgtctccttgggcggggtcttcttctgaatatagctctgtgtctcat

SEQ ID NO: 2 - protein, 214 aa, Porcine circovirus
MRHRAIFRRRPRPRRRRRHRRRYARRRLFIRRPTAGTYYTKKYSTMNVISVGTPQNNKPWHANHFITRLNEWETAISFEYYKILKMKVTLSPVISPAQQTKTMFGHTAIDLDGAWTTNTWLQDDPYAESSTRKVMTSKKKHSRYFTPKPILAGTTSAHPGQSLFFFSRPTPWLNTYDPTVQWGALLWSIYVPEKTGMTDFYGTKEVWIRYKSVL

SEQ ID NO: 3 - DNA, 891 bp, Porcine circovirus
gtccggagggaaagcccgaaacacaggtggtgttttacgataaacaactggaccccgaccgagtgggaatctattgtggagtgtggaggcagtatagcgagataccttattatcggcaaagaggttggaaaaagcggtaccccacacttgcaagggtacgtgaatttcaagaacaaaaggcgactcagctcggtgaagcgcttacccggatttggtcgggcccatctggagccggcgagggggagccacaaagaggccagcgagtattgcaagaaagagggggattacctcgagattggcgaagattcctcttcgggtaccagatcggatcttcaagcagcagctcggattctgacggagacggcgggaaatctgactgaagttgcggagaagatgcctgcagtatttatacgctatgggcggggtttgcgtgatttttgcggggtgatggggttgggtaaaccgcgtgattttaaaactgaagtttatgtttttattggtcctccaggatgcgggaaaacgcgggaagcttgtgcggatgcggctgcgcgggaattgaagctgtatttcaagccacgggggccttggtgggatggttataatggggagggtgctgttattttggatgatttttatgggtgggttccatttgatgaattgctgagaattggggacaggtaccctctgagggttcctgttaagggtgggtttgttaattttgtggctaaggtattatatattactagtaatgttgtaccggaggagtggtattcatcggagaatattcgtggaaagttggaggccttgtttaggaggttcactaaggttgtttgttggggggaggggggggtaaagaaagacatggagacagtgtatccaataaactattga

SEQ ID NO: 4 - protein, 296 aa, Porcine circovirus
VRRESPKHRWCFTINNWTPTEWESIVECGGSIARYLIIGKEVGKSGTPHLQGYVNFKNKRRLSSVKRLPGFGRAHLEPARGSHKEASEYCKKEGDYLEIGEDSSSGTRSDLQAAARILTETAGNLTEVAEKMPAVFIRYGRGLRDFCGVMGLGKPRDFKTEVYVFIGPPGCGKTREACADAAARELKLYFKPRGPWWDGYNGEGAVILDDFYGWVPFDELLRIGDRYPLRVPVKGGFVNFVAKVLYITSNVVPEEWYSSENIRGKLEALFRRFTKVVCWGEGGVKKDMETVYPINY

SEQ ID NO: 5 - DNA, 2000 bp, Porcine circovirus
tagtattacccggcacctcggaacccggatccacggaggtctgtagggagaaaaagtggtatcccattatggatgctcctcatcgtgtgagtggatataccgggcagtggatgatgaagcggcctcgtgttttgatgccgcaggacggggactggataactgagtttttgtggtgctacgagtgtcctgaagataaggacttttattgtcatcctattctaggtccggagggaaagcccgaaacacaggtggtgttttacgataaacaactggaccccgaccgagtgggaatctattgtggagtgtggaggcagtatagcgagataccttattatcggcaaagaggttggaaaaagcggtaccccacacttgcaagggtacgtgaatttcaagaacaaaaggcgactcagctcggtgaagcgcttacccggatttggtcgggcccatctggagccggcgagggggagccacaaagaggccagcgagtattgcaagaaagagggggattacctcgagattggcgaagattcctcttcgggtaccagatcggatcttcaagcagcagctcggattctgacggagacggcgggaaatctgactgaagttgcggagaagatgcctgcagtatttatacgctatgggcggggtttgcgtgatttttgcggggtgatggggttgggtaaaccgcgtgattttaaaactgaagtttatgtttttattggtcctccaggatgcgggaaaacgcgggaagcttgtgcggatgcggctgcgcgggaattgaagctgtatttcaagccacgggggccttggtgggatggttataatggggagggtgctgttattttggatgatttttatgggtgggttccatttgatgaattgctgagaattggggacaggtaccctctgagggttcctgttaagggtgggtttgttaattttgtggctaaggtattatatattactagtaatgttgtaccggaggagtggtattcatcggagaatattcgtggaaagttggaggccttgtttaggaggttcactaaggttgtttgttggggggaggggggggtaaagaaagacatggagacagtgtatccaataaactattgattttatttgcacttgtgtacaattattgcgttggggtgggggtatttattgggtgggtgggtgggcagccccctagccacggcttgtcgcccccaccgaagcatgtgggggatggggtccccacatgcgagggcgtttacctgtgcccgcacccgaagcgcagcgggagcgcgcgcgaggggacacggcttgtcgccaccggaggggtcagatttatatttatttttacttagagaacggacttgtaacgaatccaaacttctttcgtgccgtagaagtctgtcattccagttttttccgggacataaatgctccaaagcagtgctccccattgaacggtggggtcatatgtgttgagccatggggtgggtctggagaaaaagaagaggctttgtcctgggtgagcgctggtagttcccgccagaattggtttcggggtgaagtaacggctgtgtttttttttagaagtcataactttacgagtggaactttccgcataagggtcgtcttggagccaagtgtttgtggtccaggcgccgtctagatctatggctgtgtgcccgaacatagtttttgtttgctgagctggagaaattacagggctgagtgtaactttcatctttagtatcttataatattcaaagctaattgcagtttcccactcgtttaggcgggtaatgaagtggttggcgtgccagggcttattattctgaggagttccaacggaaatgacgttcatggtggagtatttctttgtgtagtatgtgccagctgtgggcctcctaatgaatagtcttcttctggcatagcgccttctgtggcgtcgtcgtctccttgggcggggtcttcttctgaatatagctctgtgtctcattttggtgccgggc

SEQ ID NO: 6 - protein, 28 aa, Artificial Sequence
  Peptide
MRHRAIFRRRPRPRRRRRHRRRYARRRL

SEQ ID NO: 7 - protein, 35 aa, Artificial Sequence
  Peptide
FIRRPTAGTYYTKKYSTMNVISVGTPQNNKPWHAN

SEQ ID NO: 8 - protein, 24 aa, Artificial Sequence
  Peptide
HFITRLNEWETAISFEYYKILKMK

SEQ ID NO: 9 - protein, 38 aa, Artificial Sequence
  Peptide
VTLSPVISPAQQTKTMFGHTAIDLDGAWTTNTWLQDDP

SEQ ID NO: 10 - protein, 10 aa, Artificial Sequence
  Peptide
YAESSTRKVM

SEQ ID NO: 11 - protein, 9 aa, Artificial Sequence
  Peptide
TSKKKHSRY

SEQ ID NO: 12 - protein, 18 aa, Artificial Sequence
  Peptide
FTPKPILAGTTSAHPGQS

SEQ ID NO: 13 - protein, 24 aa, Artificial Sequence
  Peptide
LFFFSRPTPWLNTYDPTVQWGALL

SEQ ID NO: 14 - protein, 28 aa, Artificial Sequence
  Peptide
WSIYVPEKTGMTDFYGTKEVWIRYKSVL

SEQ ID NO: 15 - protein, 27 aa, Artificial Sequence
  Peptide
VRRESPKHRWCFTINNWTPTEWESIVE

SEQ ID NO: 16 - protein, 18 aa, Artificial Sequence
  Peptide
CGGSIARYLIIGKEVGKS

SEQ ID NO: 17 - protein, 27 aa, Artificial Sequence
  Peptide
GTPHLQGYVNFKNKRRLSSVKRLPGFG

SEQ ID NO: 18 - protein, 21 aa, Artificial Sequence
  Peptide
RAHLEPARGSHKEASEYCKKE

SEQ ID NO: 19 - protein, 39 aa, Artificial Sequence
  Peptide
GDYLEIGEDSSSGTRSDLQAAARILTETAGNLTEVAEKM

SEQ ID NO: 20 - protein, 17 aa, Artificial Sequence
  Peptide
PAVFIRYGRGLRDFCGV

SEQ ID NO: 21 - protein, 14 aa, Artificial Sequence
  Peptide
MGLGKPRDFKTEVY

SEQ ID NO: 22 - protein, 33 aa, Artificial Sequence
  Peptide
VFIGPPGCGKTREACADAAARELKLYFKPRGPW

SEQ ID NO: 23 - protein, 18 aa, Artificial Sequence
  Peptide
WDGYNGEGAVILDDFYGW

SEQ ID NO: 24 - protein, 20 aa, Artificial Sequence
  Peptide
VPFDELLRIGDRYPLRVPVK

SEQ ID NO: 25 - protein, 35 aa, Artificial Sequence
  Peptide
GGFVNFVAKVLYITSNVVPEEWYSSENIRGKLEAL

SEQ ID NO: 26 - protein, 27 aa, Artificial Sequence
  Peptide
FRRFTKVVCWGEGGVKKDMETVYPINY

(19) United States
(12) Patent Application Publication
(10) Pub. No.: US 2020/0405842 A1
(43) Pub. Date: Dec. 31, 2020

(54) RECOMBINANT SWINEPOX VIRUS ENCODING A PCV3 ANTIGEN

(71) Applicant: CEVA SANTE ANIMALE, LIBOURNE (FR)

(72) Inventors: DOLORES RODRIGUEZ AGUIRRE, CANTOBLACO - MADRID (ES); MATEO DEL POZO VEGAS, VALDEOLMOS - MADRID (ES); JOSE FRANCISCO RODRIGUEZ AGUIRRE, CANTOBLACO - MADRID (ES); JUAN RAMON RODRIGUEZ FERNANDER-ALBA, VALDEOLMOS - MADRID (ES)

(21) Appl. No.: 16/770,639

(22) PCT Filed: Dec. 7, 2018
(86) PCT No.: PCT/EP2018/084029

§ 371 Date: Jun. 8, 2020

(30) Foreign Application Priority Data
Dec. 8, 2017 (EP) 17306729.9

(51) Int. Cl. A61K 39/12; C12N 15/113

(52) U.S. Cl. CPC A61K 39/12; A61K 2039/552; C12N 15/1135

(57) ABSTRACT

The present invention relates to novel recombinant swinepox viruses and their use in vaccine compositions. The recombinant swinepox viruses of the invention encode a PCV3 antigen and may be used as vaccines.

The present invention relates to novel swinepox viruses and their use. The swinepox viruses of the invention contain and express a PCV3 antigen. The SPV of the invention are suited to produce swine vaccines, particularly for vaccinating against PCV3 infection.

### BACKGROUND

Porcine Circoviruses (PCV) are responsible for major diseases in swine herds. Main serotypes of PCV identified so far were PCV1 and PCV2. Recently, a further PCV serotype has been characterized, designated PCV3, which can cause severe diseases in swine. Several strategies have been considered to treat PCV3 infections, such as inactivated PCV3 vaccines, attenuated PCV3 vaccines or subunit vaccines. No effective treatment or strategy has been provided yet against diseases caused by PCV3.

The inventors have been able to design recombinant swinepox viruses containing and expressing PCV3 antigens that can be responsible for PCV3-associated diseases. Such rSPV are safe, stable and represent potent means for inducing anti-PCV3 protective immune response in swine and for treating PCV3-associated diseases.

### SUMMARY OF THE INVENTION

The present invention relates to a recombinant swinepox virus (rSPV) comprising a nucleic acid sequence encoding a PCV3 antigen. In a preferred embodiment, the rSPV comprises an inactive endogenous gene such as an inactive IL18bp, TK, ARP and/or serpin gene.

A further object of the invention resides in a nucleic acid molecule comprising the genome of a SPV as defined above.

A further object of the invention is a host cell comprising a SPV or a nucleic acid molecule of the invention.

The present invention further provides a method for producing a rSPV, comprising infecting or introducing into a competent cell a nucleic acid molecule as defined above and collecting the rSPV.

The invention also relates to a method for propagating a rSPV, comprising infecting a competent cell with a rSPV as defined above and collecting the rSPV produced by said cells.

The invention also concerns a composition, preferably a veterinary composition, comprising a rSPV as defined above, or a cell as defined above, or a nucleic acid molecule as defined above, and an excipient.

A further object of the invention is a vaccine composition comprising a rSPV as defined above, or a cell as defined above, or a nucleic acid molecule as defined above, a suitable excipient and, optionally, an adjuvant.

The invention also relates to a rSPV or cell or nucleic acid molecule as defined above for use for treating PCV3 infection or disease in a mammal.

The invention also relates to a rSPV or cell or nucleic acid molecule as defined above for use for immunizing or vaccinating a porcine against PCV3.

The invention also relates to a method of vaccinating a mammal comprising administering to the mammal a rSPV or cell or nucleic acid molecule or composition as defined above.

The invention also relates to a method for treating PCV3 infection or associated disease in a mammal comprising administering to the mammal a rSPV or cell or nucleic acid molecule or composition as defined above.

The invention also concerns a vaccination kit for immunizing a porcine which comprises the following components:
a. an effective amount of a rSPV or vaccine as defined above, and
b. a means for administering said rSPV or vaccine to said porcine.

The invention may be used to deliver and express any PCV3 antigen to a mammal, particularly a porcine. It is particularly suited for expressing PCV2 Cap protein or an immunogenic fragment or derivative thereof to immunize or vaccinate porcine (e.g., pigs, piglets, sow).

### LEGEND TO THE FIGURES

FIG. 1. Genomic structure of rSPVs of the invention

FIG. 2. Genomic structure of rSPVs of the invention

FIG. 3. SPV insertion plasmids

FIG. 4. SPV insertion plasmids

### DETAILED DESCRIPTION OF THE INVENTION

The present invention relates to the novel recombinant swinepox viruses and the uses thereof. The viruses of the invention contain a nucleic acid encoding a PCV3 antigen and may be used to prepare vaccines suitable for treating pigs.

### PCV3 Antigen

The invention provides novel SPV encoding a PCV3 antigen. The term PCV3 antigen designates any peptide, polypeptide or protein, whether glycosylated or not, which can induce an anti-PCV3 immune response.

Within the context of the invention, a peptide typically designates a molecule comprising from 4 to 30 amino acids. A polypeptide is any amino acid polymer comprising more than 30 amino acids. The term polypeptide includes full length proteins.

Within the context of the present invention, a PCV3 antigen designates more specifically (i) a peptide or polypeptide comprising all or an immunogenic fragment of a sequence selected from SEQ ID NO: 2 or 4 or of a sequence having at least 80% identity to SEQ ID NO: 2 or 4, preferably at least 85% identity to SEQ ID NO: 2 or 4, more preferably at least 90%, at least 91%, at least 92%, at least 93%, at least 94%, at least 95%, at least 96%, at least 97%, at least 98%, or at least 99% identity to SEQ ID NO: 2 or 4, or (ii) a peptide or polypeptide encoded by a nucleic acid sequence having at least 80% identity to any one of SEQ ID NO: 1, 3 or 5 or a fragment thereof, preferably at least 85% identity to SEQ ID NO: 1, 3 or 5, more preferably at least 90%, at least 91%, at least 92%, at least 93%, at least 94%, at least 95%, at least 96%, at least 97%, at least 98%, or at least 99% identity to anyone of SEQ ID NO: 1, 3 or 5 or (iii) a derivative of a peptide or polypeptide of (i) or (ii).

SEQ ID NO: 2 is an amino acid sequence of a PCV3 Cap protein. SEQ ID NO: 1 is a nucleic acid encoding SEQ ID NO: 2.

SEQ ID NO: 4 is an amino acid sequence of a PCV3 Rep protein. SEQ ID NO: 3 is a nucleic acid encoding SEQ ID NO: 4.

SEQ ID NO: 5 is a nucleic acid sequence of a PCV3 strain.

Preferred PCV3 antigens of the invention are PCV3 cap peptides or polypeptides.

The term “immunogenic” fragment of a polypeptide designates any fragment comprising at least 4 consecutive amino acid residues of said polypeptide and capable of inducing an immune response against said polypeptide. The immunogenic fragment preferably contains an epitope of the polypeptide, such as a B-cell epitope or a T-cell epitope, which may be linear or conformational. The immunogenic fragment preferably comprises at least 5, at least 6, at least 7, at least 8, at least 9, or at least 10 consecutive amino acid residues of said polypeptide. Illustrative fragments of PCV3 cap and rep proteins are provided below:

(fragment of SEQ ID NO: 2)
MRHRAIFRRRPRPRRRRRHRRRYARRRL
(fragment of SEQ ID NO: 2)
FIRRPTAGTYYTKKYSTMNVISVGTPQNNKPWHAN
(fragment of SEQ ID NO: 2)
HFITRLNEWETAISFEYYKILKMK
(fragment of SEQ ID NO: 2)
VTLSPVISPAQQTKTMFGHTAIDLDGAWTTNTWLQDDP
(fragment of SEQ ID NO: 2)
YAESSTRKVM
(fragment of SEQ ID NO: 2)
TSKKKHSRY
(fragment of SEQ ID NO: 2)
FTPKPILAGTTSAHPGQS
(fragment of SEQ ID NO: 2)
LFFFSRPTPWLNTYDPTVQWGALL
(fragment of SEQ ID NO: 2)
WSIYVPEKTGMTDFYGTKEVWIRYKSVL
(fragment of SEQ ID NO: 4)
VRRESPKHRWCFTINNWTPTEWESIVE
(fragment of SEQ ID NO: 4)
CGGSIARYLIIGKEVGKS
(fragment of SEQ ID NO: 4)
GTPHLQGYVNFKNKRRLSSVKRLPGFG
(fragment of SEQ ID NO: 4)
RAHLEPARGSHKEASEYCKKE
(fragment of SEQ ID NO: 4)
GDYLEIGEDSSSGTRSDLQAAARILTETAGNLTEVAEKM
(fragment of SEQ ID NO: 4)
PAVFIRYGRGLRDFCGV
(fragment of SEQ ID NO: 4)
MGLGKPRDFKTEVY
(fragment of SEQ ID NO: 4)
VFIGPPGCGKTREACADAAARELKLYFKPRGPW
(fragment of SEQ ID NO: 4)
WDGYNGEGAVILDDFYGW
(fragment of SEQ ID NO: 4)
VPFDELLRIGDRYPLRVPVK
(fragment of SEQ ID NO: 4)
GGFVNFVAKVLYITSNVVPEEWYSSENIRGKLEAL
(fragment of SEQ ID NO: 4)
FRRFTKVVCWGEGGVKKDMETVYPINY

Sequence identity refers to a relationship between a given polypeptide or nucleotide sequence and a reference sequence. The level of identity is determined over the entire length of the sequences, after optimal alignment thereof, on a position-by-position basis. The total number of identical positions is then divided by the total number of residues in the sequence, resulting in a % sequence identity. Sequence identity can be readily calculated by known methods, including but not limited to, those described in Computer Analysis of Sequence Data, Part I, Griffin, A. M., and Griffin, H. G., eds., Humana Press, New Jersey (1994). Sequence identity can be determined using publicly available computer programs such as the GCG program package (Devereux, J., et al., Nucleic Acids Research, 12(1):387 (1984)), BLASTP, BLASTN, BLASTX and FASTA (Altschul, S. F. et al., J. Molec. Biol., 215:403-410(1990).

Derivatives include any variant of a peptide or polypeptide as defined above comprising 1 to 10 amino acid modifications selected from substitution, addition or insertion of 1 amino acid residues.

In a preferred embodiment, the PCV3 antigen is a peptide or polypeptide comprising all or an immunogenic fragment of SEQ ID NO: 2 or of a sequence having at least 90% identity to SEQ ID NO: 2, the immunogenic fragment comprising at least 8 consecutive amino acid residues of the sequence.

In another embodiment, the PCV3 antigen is a peptide or polypeptide comprising all or an immunogenic fragment of SEQ ID NO: 4 or of a sequence having at least 90% identity to SEQ ID NO: 2, the immunogenic fragment comprising at least 8 consecutive amino acid residues of the sequence.

The PCV3-coding nucleic acid may contain a transcriptional promoter to allow or increase expression of the encoded mRNA or polypeptide. The promoter used may be a synthetic or natural promoter, including a swinepox promoter, a poxvirus promoter, or a promoter derived from different viruses or cells such as promoters derived from eukaryotic or prokaryotic organisms. Specific examples of promoters include the vaccinia virus 7.5-kD promoter (P7.5 k) (Davison A. J. et al., J. Mol. Biol., 210(4):749-69 (1989)), 11-kD promoter (P11 k) (Bertholet et al., Proc. Nat. Acad. Sci., 82:2096-2100 (1985)) or 28-kD promoter (P28 k) (Weir J. P. & Moss B., J. Virol. 61:75-80 (1987)), or an artificial synthetic Poxvirus promoter (Ps), the thymidine kinase promoter of herpesvirus (Ross L. J., Gen. Virol. 74:371-377 (1993)), gB protein promoter (supra) of HVT or MDV, the IE promoter of human cytomegalovirus (HCMV) (Alting-Mess M. A., Nucleic Acids Res., 17:9494 (1989)), SV40 promoter (Gunning P., Proc. Natl. Acad. Sci., 84:4931-4835 (1987)), [beta] actin promoter (supra, and Kost A. T., Nucleic Acids Res., 11:8287-8301 (1983)), [beta]-globin promoter (Spitzner J. R., Nucleic Acids Res., 18:1-11 (1990)), the LTR promoter of Rous sarcoma virus (Fiek A. et al., Nucleic Acids Res., 20:1785 (1992)), and the like. In addition, promoters of the structural proteins or the essential genes of SPV can also be used.

### Recombinant SPVs

Within the context of the invention, a recombinant swinepox virus designates, generally, a swinepox virus having an artificially (e.g., recombinantly) engineered genome. rSPV include, particularly, swinepox viruses containing one or more genetic deletion(s) and/or foreign genetic material or sequence in their genome. rSPV of the invention typically comprise a SPV genome containing an inactive viral gene and a foreign genetic sequence, packaged into a SPV capsid or envelop, which may also contain a foreign protein or peptide.

rSPVs of the present invention may be prepared or obtained starting from any SPV, such as naturally occurring SPVs, or SPVs available from collections such as ATCC, CNCM, etc, or starting from recombinant SPVs. Preferably, the SPV of the invention are produced or derived from SPV kasza strain (VR-363), isolate 17077-99 (GeneBank Acc: AF410153.1), or strain VTCC/AVA/121 (GeneBank Acc: KJ725378.1). Such SPVs are available from collections or libraries, or may be cloned from their publicly available genomic sequences. Further SPV isolates may also be isolated from infected animals and used to prepare SPV of the invention.

SPVs or rSPV may be cultured or maintained or propagated in any suitable cell. For instance, rSPVs may be cultured, maintained or propagated in embryonic swine kidney cells, such as ESK-4 cells (CL-184), routinely cultured at 37.0 in 5% CO2 in Ham's F-12K medium (Gibco, Cat. No.: 21127-022) supplemented with 1% streptomycin-penicillin (Gibco, Cat. No.: 15140-122) and 5% FBS (Gibco, Cat. No.: 10437-028).

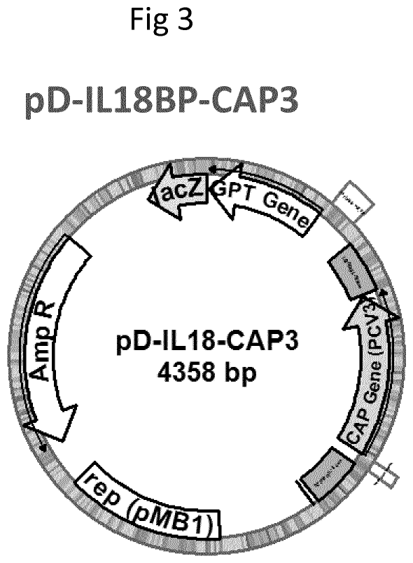

In order to construct a recombinant virus of the present invention, initially, a SPV virus (wt or recombinant) may be propagated in a suitable host cell and the SPV genomic DNA obtained. DNA can be extracted from virus-infected cells according to any conventional method. For instance, cells grown in monolayers can be scraped and then spun to harvest the supernatant. After protein is denatured in a lysis buffer and removed, DNA can be extracted with phenol and/or ethanol. Subsequently, the nucleic acid encoding a PCV3 antigen may be inserted in any suitable location of the genome. Optionally, further modification(s) of the viral genome can be made, such as insertion of another foreign nucleic acid sequence (or a cloning site allowing insertion of a foreign gene sequence), inactivation of viral genes (e.g., by mutation, deletion and/or insertion), etc. The recombinant SPV genome thus obtained may be used to produce rSPV by transformation of suitable competent cells according to conventional techniques and collection of rSPVs. Alternatively, a shuttle vector may be produced containing a PCV3 antigen-coding nucleic acid sequence (or a cloning site) flanked by sequences homologous to a target insertion region (see e.g., insertion plasmids of FIG. 3). Upon introduction into a competent cell in the presence of a SPV virus or genome, homologous recombination between the shuttle vector and the genome generates a rSPV containing the nucleic acid. Of course, once a rSPV has been engineered as described above, it can be easily replicated and propagated by simple culture on any competent cells.

In a particular embodiment, the rSPVs of the invention contain an inactive endogenous gene, which may be selected from IL18bp, TK, ARP, and/or serpin genes. In a preferred embodiment, the rSPV contains an inactive serpin gene and an inactive IL18bp gene. In another preferred embodiment, the rSPV contains an inactive serpin gene and an inactive TK gene. In a particularly preferred embodiment, the rSPV contains an inactive serpin gene, an inactive IL18bp gene, and an inactive TK gene.

Within the context of the invention, an “inactive” (or defective) gene designates a gene which has been modified and is unable to encode a functional wild-type protein or RNA encoded by the wild type gene. The gene is typically inactive as a result of a genetic alteration in the gene sequence, preferably the promoter or coding sequence, most preferably in the coding sequence. The genetic alteration may be a substitution (point mutation), addition and/or insertion of one or more nucleotides in the (coding) sequence, resulting in a reduced or total incapacity of the gene to encode a functional wild type protein/RNA. Typically, an inactive gene is a partially or fully deleted gene, typically containing a deletion of at least 20 bp within a coding sequence of said gene, preferably at least 50 bp, even more preferably at least 100 bp, further more preferably at least 150 bp, at least 200 bp, or even at least 300 bp. In a particular embodiment, the gene is inactive as a result of a full deletion of the coding sequence.

The serpin (or Serine Protease Inhibitor) gene of a viral SPV DNA contains approximately 960 bp and is generally located at nt residues 141494-142456 of a SPV genome. As a specific example, in SPV kasza strain (VR-363), the serpin gene is located at nt141494-142456. The exact position of the serpin gene in any strain of SPV may be identified easily using common knowledge, routine sequence analysis and/or sequence alignment.

Preferred rSPVs of the invention comprise an inactive serpin gene, wherein the endogenous serpin gene lacks (has been deleted of) at least 50 nt, preferably at least 100 nt, even more preferably at least 150 nt, at least 200 nt, at least 250 nt, at least 300 nt, at least 400 nt, at least 500 nt, at least 600 nt, at least 700 nt, at least 800 nt, further more preferably between 400 nt and 950 nt in the gene sequence, preferably in the coding sequence. Specific and preferred rSPVs of the invention contain a deletion of at least nt400-600 of serpin gene, even more preferably of at least nt300-700 of serpin gene, such as nt200-800 of a serpin gene. The invention shows such rSPVs can be stably propagated, have an increased cloning capacity, and have a reduced virulence.

The IL18bp gene of a viral SPV DNA contains approximately 402 bp, and is generally located at nt residues 7745-8146 of a SPV genome. As a specific example, in SPV kasza strain (VR-363), the IL18bp gene is located at nt7745-8146. The exact position of the IL18bp gene in any strain of SPV may be identified easily using common knowledge, routine sequence analysis and/or sequence alignment. Preferred rSPVs of the invention comprise an inactive IL18bp gene, wherein the endogenous IL18bp gene lacks at least 50 nt, preferably at least 100 nt, even more preferably at least 150 nt, at least 200 nt, at least 250 nt, at least 300 nt, further more preferably between 320 nt and 380 nt. Specific and preferred rSPVs of the invention contain a deletion of at least nt 100-200 of IL18bp gene, even more preferably of at least nt50-300 of IL18bp gene, such as nt31-382 or nt19-369 of IL18bp gene.

The TK gene of a viral SPV DNA contains approximately 543 bp, and is generally located at nt residues 55625-56167 of a SPV genome. As a specific example, in SPV kasza strain (VR-363), the TK gene is located at nt55625-56167. The exact position of the IL18bp gene in any strains of SPV may be identified easily using common knowledge, routine sequence analysis and/or sequence alignment. Preferred rSPVs of the invention further comprise an inactive TK gene, wherein the endogenous TK gene lacks at least 50 nt, preferably at least 100 nt, even more preferably at least 150 nt, at least 200 nt, at least 250 nt, at least 300 nt, further more preferably at least 400 nt, such as between 420 nt and 500 nt. Specific and preferred rSPVs of the invention contain a deletion of at least nt 100-300 of TK gene, even more preferably of at least nt70-450 of TK gene, even more preferably of at least nt60-500 of the TK gene, such as nt59-536 of the TK gene.

The ARP gene of a viral SPV DNA contains approximately 1455 bp, and is generally located at nt residues 137100-138554 of a SPV genome. As a specific example, in SPV kasza strain (VR-363), the ARP gene is located at nt137100-138554. The exact position of the IL18bp gene in any strains of SPV may be identified easily using common knowledge, routine sequence analysis and/or sequence alignment. As regards the ARP gene, in rSPVs of the invention comprising an inactive ARP gene, the endogenous ARP gene coding sequence preferably lacks at least 50 nt, preferably at least 100 nt, even more preferably at least 110 nt. Specific and preferred rSPVs of the invention contain a deletion of at least nt1150-1200 of ARP gene, even more preferably of at least nt1130-1220 of ARP gene, even more preferably of at least nt1116-1228 of the ARP gene. Larger deletions may also be performed, covering between 800 and 1300 bp of the ARP gene.

In a particular embodiment, the rSPVs of the invention contains a deletion of at least 100 nt in the serpin gene, a deletion of nt50-300 of IL18bp gene, and a deletion of at least nt70-450 of TK gene.

In a specific embodiment, the rSPV contains a deletion of nt31-382 or nt19-369 of IL18bp gene and a deletion of nt59-536 of the TK gene.

The nucleic acid sequence encoding the PCV3 antigen may be located in any non-essential position in the SPV genome, preferably within (or in place of) one of the above inactivated genes.

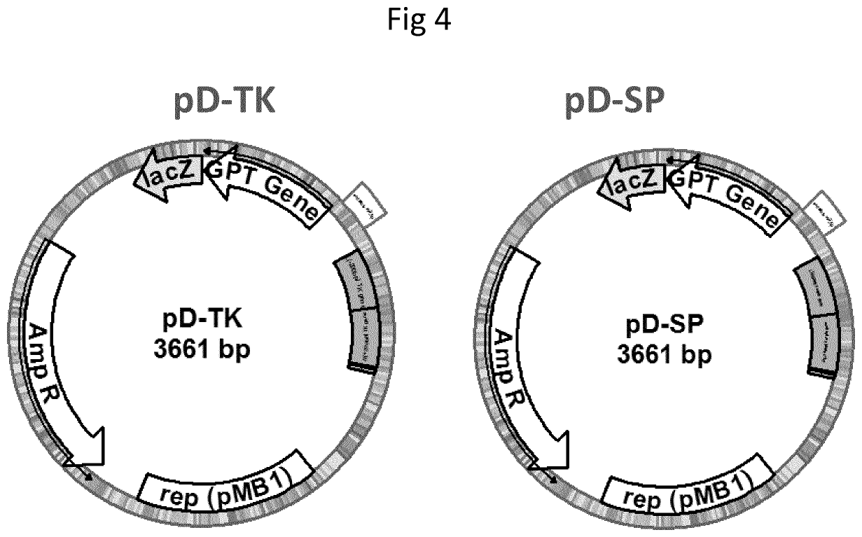

The insertion of the PCV3-coding nucleic acid sequence in the SPV genome can be performed by known methods such as mutagenesis, PCR, homologous recombination, etc. In a particular embodiment, a shuttle vector (or insertion plasmid) is prepared by recombinant DNA technology in which the PCV3-coding nucleic acid sequence is cloned flanked by two viral homology regions. The homology regions typically contain each between 50-1000 nt of a target gene sequence, allowing specific homologous recombination. The shuttle vector may be prepared from any known or conventional plasmids, cosmids, phages, and the like, such as pBS plasmids, pBR322, pUC18, pUC19 and pHC79. Examples of shuttle vectors (or insertion plasmids) are provided in FIGS. 3 and 4. The shuttle vector may then be introduced into an SPV-infected cell using known techniques such as electroporation, calcium phosphate, a lipofectin-based method, or the like. Recombinant SPV viruses having integrated the foreign nucleic acid sequence are then selected. Their sequence can be verified. The rSPV can then be maintained in any suitable competent cell. The virus can be maintained in culture, or purified and frozen or lyophilized.

In a particular embodiment, the invention relates to a rSPV which comprises (i) a nucleic acid encoding a PCV3 capsid protein or peptide inserted in the IL18bp gene, preferably in replacement of at least a portion of said IL18bp gene sequence, and (ii) optionally an inactive serpin gene and/or an inactive TK gene.

In another particular embodiment, the invention relates to a rSPV which comprises (i) a nucleic acid encoding a PCV3 capsid protein or peptide inserted in the TK gene, preferably in replacement of at least a portion of said TK gene sequence, and (ii) optionally an inactive serpin gene and/or an inactive IL18bp gene.

In another particular embodiment, the invention relates to a rSPV which comprises (i) a nucleic acid encoding a PCV3 capsid protein or peptide inserted in the SP gene, preferably in replacement of at least a portion of said SP gene sequence, and (ii) optionally an inactive TK gene and/or an inactive IL18bp gene.

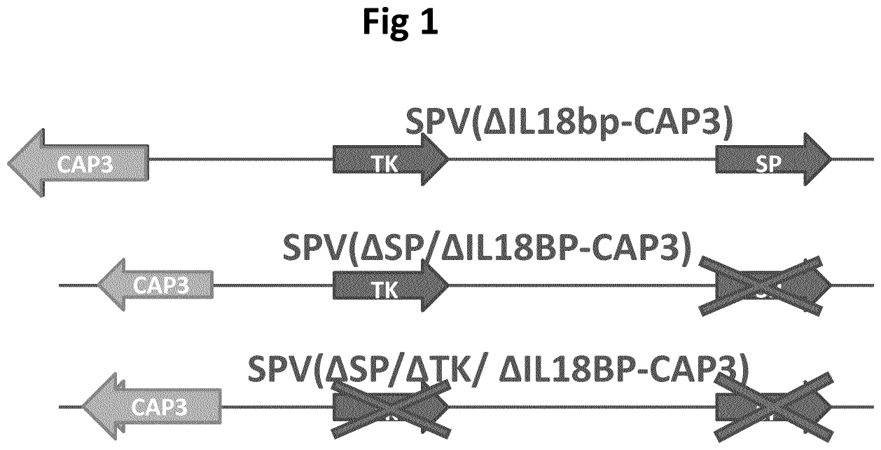

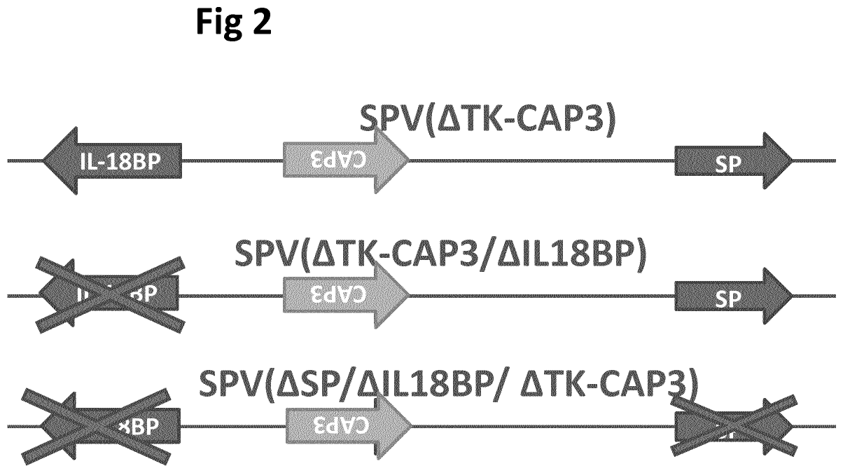

Specific examples of rSPVs of the invention are disclosed FIGS. 1 and 2.

### Additional Foreign Nucleic Acid Sequence

The rSPVs of the invention may contain one or more additional foreign nucleic acid sequences, typically foreign gene sequences encoding an mRNA, a peptide or a polypeptide (or protein). The foreign gene sequence may, for instance, encode various types of active molecules, such as an antigen, adjuvant, cytokine, lymphokine, growth factor, enzyme, label, etc.

In a preferred embodiment, the additional foreign gene sequence encodes an antigen (peptide, polypeptide or protein antigen) from a pathogen of a porcine infectious disease, and most preferably an antigen from a virus, bacterium, fungus, or protozoa.

The additional foreign gene sequence preferably encodes a peptide or polypeptide (e.g., glycoprotein, capsid protein, or fragment thereof) of a virus or pathogen selected from porcine circovirus (PCV1, PCV2, PCV2a, PCV2b, PCV2d), Actinobacillus pleuropneunomia; Adenovirus; Alphavirus such as Eastern equine encephalomyelitis viruses; Balantidium coli; Bordetella bronchiseptica; Brachyspira spp., preferably B. hyodyentheriae, B. pilosicoli, B. innocens, Brucella suis, preferably biovars 1, 2 and 3; Classical swine fever virus, African swine fever virus; Chlamydia and Chlamydophila sp. and preferably C. pecorum and C. abortus; Clostridium spp., preferably Cl. difficile, Cl. perfringens types A, B and C, Cl. novyi, Cl. septicum, Cl. tetani; Digestive and respiratory Coronavirus; Cryptosporidium parvum; Eimeria spp; Eperythrozoonis suis currently named Mycoplasma haemosuis; Erysipelothrix rhusiopathiae; Escherichia coli; Haemophilus parasuis, preferably subtypes 1, 7 and 14; Hemagglutinating encephalomyelitis virus; lsospora suis; Japanese Encephalitis virus; Lawsonia intracellulars; Leptospira spp., preferably Leptospira australis, Leptospira canicola, Leptospira grippotyphosa, Leptospira icterohaemorrhagicae, Leptospira interrogans, Leptospira Pomona and Leptospira tarassovi; Mannheimia haemolytica; Mycobacterium spp. preferably, M. avium, M. intracellular and M. bovis: Mycoplasma hyponeumoniae; Parvovirus; Pasteurella multocida; Porcine cytomegolovirus; Porcine parovirus, Porcine reproductive and respiratory syndrome virus: Pseudorabies virus; Rotavirus; Sagiyama virus; Salmonella spp. preferably, S. thyhimurium and S. choleraesuis; Staphylococcus spp. preferably, S. hyicus; Streptococcus spp., preferably Strep, suis; Swine cytomegalovirus; Swine herpes virus; Swine influenza virus; Swinepox virus; Toxoplasma gondii; Vesicular stomatitis virus or virus of exanthema of swine.

In a particularly preferred embodiment, the additional foreign gene sequence encodes a PCV2 antigen, particularly a PCV2 protein or peptide, even more particularly a PCV2 capsid (e.g., ORF2) protein or peptide.

The foreign gene sequence may contain a transcriptional promoter to allow or increase expression of the encoded mRNA or polypeptide. The promoter used may be a synthetic or natural promoter, including a swinepox promoter, a poxvirus promoter, or a promoter derived from different viruses or cells such as promoters derived from eukaryotic or prokaryotic organisms. Specific examples of promoters include the vaccinia virus 7.5-kD promoter (P7.5 k) (Davison A. J. et al., J. Mol. Biol., 210(4):749-69 (1989)), 11-kD promoter (P11 k) (Bertholet et al., Proc. Nat. Acad. Sci., 82:2096-2100 (1985)) or 28-kD promoter (P28 k) (Weir J. P. & Moss B., J. Virol. 61:75-80 (1987)), or an artificial synthetic Poxvirus promoter (Ps), the thymidine kinase promoter of herpesvirus (Ross L. J., Gen. Virol. 74:371-377 (1993)), gB protein promoter (supra) of HVT or MDV, the IE promoter of human cytomegalovirus (HCMV) (Alting-Mess M. A., Nucleic Acids Res., 17:9494 (1989)), SV40 promoter (Gunning P., Proc. Natl. Acad. Sci., 84:4931-4835 (1987)), [beta] actin promoter (supra, and Kost A. T., Nucleic Acids Res., 11:8287-8301 (1983)), [beta]-globin promoter (Spitzner J. R., Nucleic Acids Res., 18:1-11 (1990)), the LTR promoter of Rous sarcoma virus (Fiek A. et al., Nucleic Acids Res., 20:1785 (1992)), and the like. In addition, promoters of the structural proteins or the essential genes of SPV can also be used.

The PCV3-coding nucleic acid and the additional foreign gene sequence(s) may be located in a same cloning region and/or in distinct cloning sites. Also, they may be under the control of the same or distinct promoters, and in the same or opposite orientation.

### Nucleic Acid Molecules

The invention also relates to nucleic acid molecules comprising the genome of a rSPV of the invention. Nucleic acid molecules of the invention may be DNA or RNA, double-stranded or single-stranded. Single-stranded DNA or RNA may be the coding strand, also known as the sense strand, or it may be the non-coding strand, also referred to as the anti-sense strand. The invention also relates to variants or analogs of such nucleic acid molecules, e.g., molecules having at least 85%, 90%, 95%, 96%, 97%, 98% or more sequence identity thereof.

The degree of homology between two nucleic acid sequences may be determined by means of computer programs known in the art such as GAP provided in the GCG program package (Program Manual for the Wisconsin Package, Version 8, August 1996, Genetics Computer Group, 575 Science Drive, Madison, Wis., USA 5371 1) (Needleman, S. B. and Wunsch, C D., (1970), Journal of Molecular Biology, 48, 443-453). Using GAP with the following settings for DNA sequence comparison: GAP creation penalty of 5.0 and GAP extension penalty of 0.3. Nucleic acid molecules may be aligned to each other using the Pileup alignment software, available as part of the GCG program package, using, for instance, the default settings of gap creation penalty of 5 and gap width penalty of 0.3.

Suitable experimental conditions for determining whether a given nucleic acid molecule hybridizes to a specified nucleic acid may involve pre-soaking of a filter containing a relevant sample of the nucleic acid to be examined in 5×SSC for 10 minutes, and pre-hybridization of the filter in a solution of 5×SSC, 5×Denhardt's solution, 0.5% SDS and 100 [mu]g/ml of denatured sonicated salmon sperm DNA, followed by hybridization in the same solution containing a concentration of 10 ng/ml of a P-dCTP-labeled probe for 12 hours at approximately 45° C., in accordance with the hybridization methods as described in Sambrook et al. (1989; Molecular Cloning, A Laboratory Manual, 2nd edition, Cold Spring Harbour, New York). The filter is then washed twice for 30 minutes in 2×SSC, 0.5% SDS at least 55° C. (low stringency), at least 60° C. (medium stringency), at least 65° C. (medium/high stringency), at least 70° C. (high stringency), or at least 75° C. (very high stringency). Hybridization may be detected by exposure of the filter to an x-ray film.

The nucleic acid molecules according to the invention may be provided in the form of a nucleic acid molecule per se such as naked nucleic acid molecules; a vector; virus or host cell etc. Vectors include expression vectors that contain a nucleic acid molecule of the invention.

### Host Cells

In a further embodiment of the invention, there is provided a host cell transformed with a nucleic acid or with a rSPV according to the invention. Such cells can produce rSPVs of the invention. Suitable examples of host cells are known to those skilled in the art or can be readily selected by those skilled in the art. Host cells are preferably eukaryotic cells such as mammalian (e.g., pig), fungal (e.g. Saccharomyces cerevisiae, pichia, aspergillus, fusarium), insect and plant cells. Specific examples of host cells are swine kidney cells, such as ESK-4 cells (CL-184).

### Vaccine Compositions and Methods

The term “vaccine” as used herein includes any composition which may be used to cause, stimulate or amplify an immune response in an animal (e.g., pigs) against a pathogen. Particular examples of vaccines of the invention are composition able to cause or stimulate or amplify immunity against a PCV3 virus. In a vaccine of the invention, the at least one foreign gene sequence shall encode an antigen or an adjuvant.

The term “immunization” includes the process of delivering an immunogen to a subject. Immunization may, for example, enable a continuing high level of antibody and/or cellular response in which T-lymphocytes can kill or suppress the pathogen in the immunized non-human animal, such as pig, which is directed against a pathogen or antigen to which the animal has been previously exposed.

Vaccines of the invention comprise an immunologically effective amount of a rSPV or nucleic acid or cell as described above in a pharmaceutically acceptable vehicle.

In practice, the exact amount required for an immunologically effective dose may vary from subject to subject depending on factors such as the age and general condition of the subject, the nature of the formulation and the mode of administration. Appropriate “effective amount” may be determined by one of ordinary skill in the art using only routine experimentation. For instance, methods are known in the art for determining or titrating suitable dosages of a vaccine to find minimal effective dosages based on the weight of the non-human animal subject, concentration of the vaccine and other typical factors. In a typical embodiment, the vaccine comprises a unitary dose of between 10 and 10,000,000 TCID₅₀, preferably between 100 and 1,000,000 TCID₅₀, even more preferably of between 1,000 and 100,000 TCID₅₀, of a rSPV of the invention. TCID₅₀ designates the median tissue culture infective dose, i.e., the amount of virus that produces pathological change in 50% of inoculated cell cultures.

The dosage of the vaccine, concentration of components therein and timing of administering the vaccine, which elicit a suitable immune response, can be determined by methods such as by antibody titrations of sera, e.g., by ELISA and/or seroneutralization assay analysis and/or by vaccination challenge evaluation.

Vaccines may comprise other ingredients, known per se by one of ordinary skill in the art, such as pharmaceutically acceptable carriers, excipients, diluents, adjuvants, freeze drying stabilizers, wetting or emulsifying agents, pH buffering agents, gelling or viscosity enhancing additives, or preservatives, depending on the route of administration.

Examples of pharmaceutically acceptable carriers, excipients or diluents include, but are not limited to demineralised or distilled water; saline solution; vegetable based oils such as peanut oil, arachis oil, safflower oil, olive oil, cottonseed oil, maize oil, sesame oil, or coconut oil; silicone oils, including polysiloxanes, such as methyl polysiloxane, phenyl polysiloxane and methylphenyl polysolpoxane; volatile silicones; mineral oils such as light liquid paraffin oil, or heavy liquid paraffin oil; squalene; cellulose derivatives such as methyl cellulose, ethyl cellulose, carboxymethylcellulose, carboxymethylcellulose sodium salt, or hydroxypropyl methylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrrolidone; agar; carrageenan; gum tragacanth or gum acacia, and petroleum jelly. Typically, the carrier or carriers will form from 10% to 99.9% by weight of the vaccine composition and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

Examples of adjuvants include, but are not limited to, oil in water emulsions, aluminum hydroxide (alum), immunostimulating complexes, non-ionic block polymers or copolymers, cytokines (like IL-1, IL-2, IL-7, IFN-[alpha], IFN-[beta], IFN-γ, etc.), saponins, monophosphoryl lipid A (MLA), muramyl dipeptides (MDP) and the like. Other suitable adjuvants include, for example, aluminum potassium sulfate, heat-labile or heat-stable enterotoxin(s) isolated from Escherichia coli, cholera toxin or the B subunit thereof, diphtheria toxin, tetanus toxin, pertussis toxin, Freund's incomplete or complete adjuvant, etc. Toxin-based adjuvants, such as diphtheria toxin, tetanus toxin and pertussis toxin may be inactivated prior to use, for example, by treatment with formaldehyde.

Examples of freeze-drying stabilizer may be for example carbohydrates such as sorbitol, mannitol, starch, sucrose, dextran or glucose, proteins such as albumin or casein, and derivatives thereof.

The vaccine compositions of the invention may be liquid formulations such as an aqueous solution, water-in-oil or oil-in-water emulsion, syrup, an elixir, a tincture, a preparation for parenteral, subcutaneous, intradermal, intramuscular or intravenous administration (e.g., injectable administration), such as sterile suspensions or emulsions. Such formulations are known in the art and are typically prepared by dissolution of the antigen and other typical additives in the appropriate carrier or solvent systems. Liquid formulations also may include suspensions and emulsions that contain suspending or emulsifying agents.

The route of administration can be percutaneous, via mucosal administration, or via a parenteral route (intradermal, intramuscular, subcutaneous, intravenous, or intraperitoneal). The vaccine of the invention can conveniently be administered intranasally, transdermally (i.e., applied on or at the skin surface for systemic absorption), parenterally, ocularly, etc. The parenteral route of administration includes, but is not limited to, intramuscular, intravenous, intraperitoneal routes and the like.

The vaccines of the invention can be administered as single doses or in repeated doses. The vaccines of the invention can be administered alone, or can be administered simultaneously or sequentially administered with one or more further compositions, such as for example other porcine immunogenic or vaccine compositions. Where the compositions are administered at different times the administrations may be separate from one another or overlapping in time.

The present invention also relates to methods of immunizing or inducing an immune response in a non-human mammal (e.g., pigs) comprising administering to said mammal a rSPV or a nucleic acid, or a cell or a vaccine as described above.

Vaccines of the invention are preferably administered to pigs, adult pigs, but also to young pigs, piglets or to pregnant sow. Vaccination of pregnant sows is advantageous as it can confer passive immunity to the newborns via the transmission of maternal antibodies. The pigs may be less than 7, 6, 5, 4, 3, 2 or 1 week old; 1 to 6 weeks old; 2 to 5 weeks old; or 3 to 4 weeks old. Desirably, the vaccine is administered to a subject who has not yet been exposed to the pathogen.

The present invention also provides a container comprising an immunologically effective amount a rSPV, nucleic acid, cell or vaccine as described above. The invention also provides vaccination kits comprising an optionally sterile container comprising an immunologically effective amount of the vaccine, means for administering the vaccine to animals, and optionally an instruction manual including information for the administration of the immunologically effective amount the composition for treating and/or preventing infectious disease.

The invention is particularly suited for the treatment (preventive and curative) of PCV3 infection and associated diseases.

A further aspect of the invention relates to methods of treating and/or preventing a PCV3 associated disease in a non-human mammal, and to methods of immunizing or vaccinating a non-human animal subject, such as pigs, swine, sow, piglet, against PCV3 infection, comprising administering to said animal subject a rSPV, a nucleic acid, a cell, or vaccine composition as defined above.

PCV3 infections or associated diseases include inter alia Postweaning Multisystemic Wasting Syndrome (PMWS), Porcine Dermatitis and Nephropathy Syndrome (PDNS), Porcine Respiratory Disease Complex (PRDC), reproductive disorders, granulomatous enteris, exsudative epidermitis, necrotizing lymphadenitis, and congenital tremors. Preferably, a non-human animal subject, such as pig, is protected to an extent in which one to all of the adverse physiological symptoms or effects of PCV3 infections are significantly reduced, ameliorated or totally prevented.

In one embodiment, the vaccine compositions of the invention are administered to a pig susceptible to or otherwise at risk for PCV3 infection to enhance the subject own immune response capabilities.

Preferably, the subject is a pig which is in need of vaccination against Postweaning Multisystemic Wasting Syndrome (PMWS) and/or Porcine Dermatitis and Nephropathy Syndrome (PDNS).

Further aspects and advantages of the invention shall be disclosed in the following experimental section, which illustrates the claimed invention.

### Examples

SPV kasza strain (VR-363) and embryonic swine kidney cell, ESK-4 cells (CL-184) could be purchased from the American Type Culture Collection (ATCC). The ESK-4 cells are routinely cultured at 37.0 in 5% CO2 in Ham's F-12K medium (Gibco, Cat. No.: 21127-022) supplemented with 1% streptomycin-penicillin (Gibco, Cat. No.: 15140-122) and 5% FBS (Gibco, Cat. No.: 10437-028). For SPV genomic DNA preparation, confluent ESK-4 cells in a 225 cm2 flask can be infected with SPV and incubated for 6 days until the cells show 100% cytopathic effect (CPE). The infected cells can then be harvested by scraping the cells into the medium and centrifuging at 1300 rpm for 5 min. The medium is decanted, and the cell pellet is gently resuspended in 2 ml Phosphate Buffer Saline (PBS: 1.5 g Na2HPO4, 0.2 g KH2PO4, 0.8 g NaCl and 0.2 g KCl per litter H2O) and subjected to two successive freeze-thaws. Cellular debris are then removed by centrifuging at 3000 rpm for 5 min at 4° C. SPV virions, present in supernatant, are then pelleted by centrifugation at 20,000×g for 20 min at 4° C. The resultant pellets are then suspended with 10 mM Tris pH7.5. SPV genomic DNAs are then extracted from the SPV virions by suspending with the lysis buffer (20 mM Tris, pH9, 0.1M NaCl2, 5 mM EDTA, 0.1% SDS, 0.2 mg/ml proteinase K) and incubating at 60.0 for 5 min. Phenol:chlororoform (1:1) extraction is conducted two times, and the sample precipitated by the addition of two volumes of ethanol and centrifugation. The supernatant is decanted, and the pellet (SPV DNA) is air dried and rehydrated in 10 mM Tris pH7.5, 1 mM EDTA at 4° C.

The flanking regions of serpin (SP) gene and of TK gene in the SPV genome were cloned by Polymerase Chain Reaction (PCR) and used to produce insertion plasmids (FIG. 4).

The flanking regions of IL18BP gene in the SPV genome were cloned by Polymerase Chain Reaction (PCR) and used to produce insertion plasmid (FIG. 3).

Recombinant SPVs are generated in ESK-4 cells by homologous recombination between wild-type SPV genome and insertion plasmid vectors. Sub-confluent ESK-4 cells in a 6-well plate are infected with wild-type SPV (wtSPV) or with a recombinant SPV having an inactive IL18bp gene and/or an inactive TK gene, and 17 hr later the infected cells are transfected with 2 μg of pD-SP using Lipofectamin Plus reagent (Invitrogen) and allowed to incubate at 37.0 for 5 days until cytopathic effect (CPE) has occurred. Cell lysates from infected-transfected cells can be collected and transferred to new blank 96-well plates, and infected cells lysed with lysis buffer (20 mM Tris-Cl, 0.1M NaCl, 5 mM EDTA, 0.1% SDS, 200 μg/ml protenase K) followed by heat treatment (60° C. 5 min, and 98° C. 2 min). The rSPVs are isolated from said lysate. The genomic structure of different rSPVs is represented FIGS. 1 and 2.

List of sequences

SEQ ID NO: 1: PCV3 Cap (antisense)
TTAGAGAACGGACTTGTAACGAATCCAAACTTCTTTCGTGCCGTAGAAG
TCTGTCATTCCAGTTTTTTCCGGGACATAAATGCTCCAAAGCAGTGCTC
CCCATTGAACGGTGGGGTCATATGTGTTGAGCCATGGGGTGGGTCTGGA
GAAAAAGAAGAGGCTTTGTCCTGGGTGAGCGCTGGTAGTTCCCGCCAGA
ATTGGTTTCGGGGTGAAGTAACGGCTGTGTTTTTTTTTAGAAGTCATAA
CTTTACGAGTGGAACTTTCCGCATAAGGGTCGTCTTGGAGCCAAGTGTT
TGTGGTCCAGGCGCCGTCTAGATCTATGGCTGTGTGCCCGAACATAGTT
TTTGTTTGCTGAGCTGGAGAAATTACAGGGCTGAGTGTAACTTTCATCT
TTAGTATCTTATAATATTCAAAGCTAATTGCAGTTTCCCACTCGTTTAG
GCGGGTAATGAAGTGGTTGGCGTGCCAGGGCTTATTATTCTGAGGAGTT
CCAACGGAAATGACGTTCATGGTGGAGTATTTCTTTGTGTAGTATGTGC
CAGCTGTGGGCCTCCTAATGAATAGTCTTCTTCTGGCATAGCGCCTTCT
GTGGCGTCGTCGTCTCCTTGGGCGGGGTCTTCTTCTGAATATAGCTCTG
TGTCTCAT
SEQ ID NO: 2: PCV3 Cap
MRHRAIFRRRPRPRRRRRHRRRYARRRLFIRRPTAGTYYTKKYSTMNVI
SVGTPQNNKPWHANHFITRLNEWETAISFEYYKILKMKVTLSPVISPAQ
QTKTMFGHTAIDLDGAWTTNTWLQDDPYAESSTRKVMTSKKKHSRYFTP
KPILAGTTSAHPGQSLFFFSRPTPWLNTYDPTVQWGALLWSIYVPEKTG
MTDFYGTKEVWIRYKSVL
SEQ ID NO: 3: PCV3 Rep
GTCCGGAGGGAAAGCCCGAAACACAGGTGGTGTTTTACGATAAACAACT
GGACCCCGACCGAGTGGGAATCTATTGTGGAGTGTGGAGGCAGTATAGC
GAGATACCTTATTATCGGCAAAGAGGTTGGAAAAAGCGGTACCCCACAC
TTGCAAGGGTACGTGAATTTCAAGAACAAAAGGCGACTCAGCTCGGTGA
AGCGCTTACCCGGATTTGGTCGGGCCCATCTGGAGCCGGCGAGGGGGAG
CCACAAAGAGGCCAGCGAGTATTGCAAGAAAGAGGGGGATTACCTCGAG
ATTGGCGAAGATTCCTCTTCGGGTACCAGATCGGATCTTCAAGCAGCAG
CTCGGATTCTGACGGAGACGGCGGGAAATCTGACTGAAGTTGCGGAGAA
GATGCCTGCAGTATTTATACGCTATGGGCGGGGTTTGCGTGATTTTTGC
GGGGTGATGGGGTTGGGTAAACCGCGTGATTTTAAAACTGAAGTTTATG
TTTTTATTGGTCCTCCAGGATGCGGGAAAACGCGGGAAGCTTGTGCGGA
TGCGGCTGCGCGGGAATTGAAGCTGTATTTCAAGCCACGGGGGCCTTGG
TGGGATGGTTATAATGGGGAGGGTGCTGTTATTTTGGATGATTTTTATG
GGTGGGTTCCATTTGATGAATTGCTGAGAATTGGGGACAGGTACCCTCT
GAGGGTTCCTGTTAAGGGTGGGTTTGTTAATTTTGTGGCTAAGGTATTA
TATATTACTAGTAATGTTGTACCGGAGGAGTGGTATTCATCGGAGAATA
TTCGTGGAAAGTTGGAGGCCTTGTTTAGGAGGTTCACTAAGGTTGTTTG
TTGGGGGGAGGGGGGGGTAAAGAAAGACATGGAGACAGTGTATCCAATA
AACTATTGA
SEQ ID NO: 4: PCV3 Rep
VRRESPKHRWCFTINNWTPTEWESIVECGGSIARYLIIGKEVGKSGTPH
LQGYVNFKNKRRLSSVKRLPGFGRAHLEPARGSHKEASEYCKKEGDYLE
IGEDSSSGTRSDLQAAARILTETAGNLTEVAEKMPAVFIRYGRGLRDFC
GVMGLGKPRDFKTEVYVFIGPPGCGKTREACADAAARELKLYFKPRGPW
WDGYNGEGAVILDDFYGWVPFDELLRIGDRYPLRVPVKGGFVNFVAKVL
YITSNVVPEEWYSSENIRGKLEALFRRFTKVVCWGEGGVKKDMETVYPI
NY
SEQ ID NO: 5: PCV3-US/MO2015 full length nt
sequence
tagtattacc cggcacctcg gaacccggat
ccacggaggt ctgtagggag aaaaagtggt
atcccattat ggatgctcct catcgtgtga
gtggatatac cgggcagtgg atgatgaagc
ggcctcgtgt tttgatgccg caggacgggg
actggataac tgagtttttg tggtgctacg
agtgtcctga agataaggac ttttattgtc
atcctattct aggtccggag ggaaagcccg
aaacacaggt ggtgttttac gataaacaac
tggaccccga ccgagtggga atctattgtg
gagtgtggag gcagtatagc gagatacctt
attatcggca aagaggttgg aaaaagcggt
accccacact tgcaagggta cgtgaatttc
aagaacaaaa ggcgactcag ctcggtgaag
cgcttacccg gatttggtcg ggcccatctg
gagccggcga gggggagcca caaagaggcc
agcgagtatt gcaagaaaga gggggattac
ctcgagattg gcgaagattc ctcttcgggt
accagatcgg atcttcaagc agcagctcgg
attctgacgg agacggcggg aaatctgact
gaagttgcgg agaagatgcc tgcagtattt
atacgctatg ggcggggttt gcgtgatttt
tgcggggtga tggggttggg taaaccgcgt
gattttaaaa ctgaagttta tgtttttatt
ggtcctccag gatgcgggaa aacgcgggaa
gcttgtgcgg atgcggctgc gcgggaattg
aagctgtatt tcaagccacg ggggccttgg
tgggatggtt ataatgggga gggtgctgtt
attttggatg atttttatgg gtgggttcca
tttgatgaat tgctgagaat tggggacagg
taccctctga gggttcctgt taagggtggg
tttgttaatt ttgtggctaa ggtattatat
attactagta atgttgtacc ggaggagtgg
tattcatcgg agaatattcg tggaaagttg
gaggccttgt ttaggaggtt cactaaggtt
gtttgttggg gggagggggg ggtaaagaaa
gacatggaga cagtgtatcc aataaactat
tgattttatt tgcacttgtg tacaattatt
gcgttggggt gggggtattt attgggtggg
tgggtgggca gccccctagc cacggcttgt
cgcccccacc gaagcatgtg ggggatgggg
tccccacatg cgagggcgtt tacctgtgcc
cgcacccgaa gcgcagcggg agcgcgcgcg
aggggacacg gcttgtcgcc accggagggg
tcagatttat atttattttt acttagagaa
cggacttgta acgaatccaa acttctttcg
tgccgtagaa gtctgtcatt ccagtttttt
ccgggacata aatgctccaa agcagtgctc
cccattgaac ggtggggtca tatgtgttga
gccatggggt gggtctggag aaaaagaaga
ggctttgtcc tgggtgagcg ctggtagttc
ccgccagaat tggtttcggg gtgaagtaac
ggctgtgttt ttttttagaa gtcataactt
tacgagtgga actttccgca taagggtcgt
cttggagcca agtgtttgtg gtccaggcgc
cgtctagatc tatggctgtg tgcccgaaca
tagtttttgt ttgctgagct ggagaaatta
cagggctgag tgtaactttc atctttagta
tcttataata ttcaaagcta attgcagttt
cccactcgtt taggcgggta atgaagtggt
tggcgtgcca gggcttatta ttctgaggag
ttccaacgga aatgacgttc atggtggagt
atttctttgt gtagtatgtg ccagctgtgg
gcctcctaat gaatagtctt cttctggcat
agcgccttct gtggcgtcgt cgtctccttg
ggcggggtct tcttctgaat atagctctgt
gtctcatttt ggtgccgggc

1-21. (canceled)

22. A recombinant swinepox virus (rSPV) comprising at least a first foreign nucleic acid encoding a porcine circovirus serotype 3 (PCV3) antigen.

23. The rSPV of claim 22, wherein the PCV3 antigen is selected from a PCV3 capsid protein or peptide, a PCV3 REP protein or peptide, or an immunogenic fragment or derivative thereof.

24. The rSPV of claim 23, wherein the PCV3 antigen is a PCV3 capsid protein comprising SEQ ID NO: 2 or a sequence having at least 80% amino acid identity to SEQ ID NO: 2, or an immunogenic fragment or derivative thereof.

25. The rSPV of claim 23, wherein the PCV3 antigen is a PCV3 REP protein comprising SEQ ID NO: 4 or a sequence having at least 80% amino acid identity to SEQ ID NO: 4, or an immunogenic fragment or derivative thereof.

26. The rSPV of claim 22, which further comprises at least a second foreign nucleic acid encoding an antigen.

27. The rSPV of claim 22, which comprises at least one inactive viral gene selected from the IL18bp gene, the thymidine kinase (TK) gene, the ankirin repeat protein (ARP) gene, and the serpin (SP) gene.

28. The rSPV of claim 27, which comprises at least 2 inactive viral genes selected from the IL18bp gene, the thymidine kinase (TK) gene, the ankirin repeat protein (ARP) gene, and the serpin (SP) gene.

29. The rSPV of claim 22, wherein the nucleic acid encoding a PCV3 antigen is located in an insertion site selected from the IL18bp gene, the thymidine kinase (TK) gene, the ankirin repeat protein (ARP) gene, or the serpin (SP) gene.

30. The rSPV of claim 29, wherein said rSPV comprises a deletion of at least 50 bp in the IL18bp, TK, SP and/or ARP gene sequence, and wherein the nucleic acid encoding a PCV3 antigen is located in said deletion.

31. The rSPV of claim 22, which comprises (i) a nucleic acid encoding a PCV3 capsid protein or peptide inserted in the IL18bp gene, and (ii) optionally an inactive serpin gene and/or an inactive TK gene.

32. The rSPV of claim 22, which comprises (i) a nucleic acid encoding a PCV3 capsid protein or peptide inserted in the TK gene, and (ii) optionally an inactive serpin gene and/or an inactive IL18bp gene.

33. The rSPV of claim 22, wherein the first foreign nucleic acid contains a transcriptional promoter.

34. The rSPV of claim 33, wherein the promoter is selected from the vaccinia virus 7.5-kD promoter (P7.5 k), 11-kD promoter (P11 k), or 28-kD promoter (P28 k), an artificial synthetic Poxvirus promoter (Ps), the chicken beta-actin (Bac) promoter or a derivative thereof, the Pec promoter, the Murine Cytomegalovirus (Mcmv) immediate-early (ie)1 promoter, the Human Cytomegalovirus promoter (Hcmv), the Simian virus (SV)40 promoter, and the Raus Sarcoma virus (RSV) promoter, or fragments thereof which retain a promoter activity.

35. A nucleic acid molecule comprising the genome of a rSPV of claim 22.

36. A host cell comprising a rSPV of claim 22 or a nucleic acid molecule encoding said rSPV.

37. A method for producing a rSVP, comprising infecting a competent cell with a nucleic acid molecule of claim 35 and collecting the rSVP.

38. A composition comprising a rSVP of claim 22 and an excipient.

39. The composition of claim 38, which is a vaccine composition.

40. A method of immunizing a porcine comprising administering a rSPV of claim 22 to said porcine.

41. A method of treating Postweaning Multisystemic Wasting Syndrome comprising administering a rSPV of claim 22 to a porcine, swine or piglet in an amount effective to treat said porcine, swine or piglet.

42. A vaccination kit for immunizing a porcine which comprises the following components:
a) an effective amount of a vaccine composition of claim 39, and
b) a means for administering said vaccine to said porcine.
